(19) United States
(12) Patent Application Publication
(10) Pub. No.: US 2005/0079607 A1
(43) Pub. Date: Apr. 14, 2005

(54) Regulation of human pluripotential cells by bone morphogenetic protein 2 antagonists

(76) Inventors: Jessica Maryann Andrade, Croydon Hills (AU); Martin F. Pera, Prahan (AU); Daniella Herszfeld, East St. Kilda (AU)

Correspondence Address: OPPEDAHL AND LARSON LLP, P O BOX 5068, DILLON, CO, 80435-5068, US

(21) Appl. No.: 10/616,682

(22) Filed: Jul. 9, 2003

Related U.S. Application Data
(63) Continuation of application No. 09/670,198, filed on Sep. 25, 2000, now abandoned.

(51) Int. Cl. C12N 5/08

(52) U.S. Cl. 435/366; 435/404

(57) ABSTRACT

The present invention relates to regulation of the growth and/or differentiation of pluripotential cells by factors capable of inhibiting an activity of bone morphogenetic protein-2.

The present invention relates to a method of controlling the growth and/or differentiation of pluripotential cells.

It is known in the prior art that in the mouse, permanent, diploid embryonic stem cell lines may be derived from the blastocyst (embryonic stem (ES) cells) or from primordial germ cells (embryonic germ (EG) cells). Two key properties of mouse ES cells account for their revolutionary impact on modern biology: first, ES cells may be propagated indefinitely and genetically manipulated in vitro; second, when reintroduced into a host blastocyst, they participate in normal development and colonise all tissues of the host embryo, including the germ line, thus enabling the creation of new mouse strains with specific genetic modifications. ES cells also provide a model for in vitro study of peri-implantation development, and they can undergo directed differentiation into specific cell lineages, for instance to yield haematopoietic progenitor cells or adipocytes.

The availability of ES or EG cells from species of commercial importance would facilitate the creation of animals with desired characteristics. The recent establishment of human ES cells has provided a powerful tool for molecular and cellular studies of early human development, a process that is otherwise inaccessible to investigation. There are many significant practical uses for human ES cells as well. For example, human ES cell lines could serve in functional genomics applications, as in vitro screens for factors inducing differentiation and proliferation of specific embryonic or foetal precursor cell populations. If directed differentiation of human ES cells in vitro could be achieved, then the cultured cells could serve as a renewable source of committed progenitor cells for use in transplantation.

While little is known regarding the control of pluripotent stem cells in primates, the existing data suggests that present understanding of mouse ES cell regulation will be of limited use in extrapolating to humans. Mouse ES cells were derived on the basis of previous work using teratocarcinomas, embryonal neoplasms whose stem cells share many properties of ES cells, including the ability to contribute extensively to tissues of chimeric mice. It is clear that human pluripotent stem cells differ in phenotype and growth requirements from their murine counterparts. Specifically, while the cytokine leukaemia inhibitory factor, or LIF, will substantially replace the requirement for a feeder cell layer in the continuous growth of many mouse ES cell strains, human stem cells will not respond to LIF, though they both require feeder layers for continuous growth in vitro.

The difficulty of establishing ES cells in many mouse strains, the requirement for feeder cell support in addition to LIF in the establishment and maintenance of mouse ES cells observed by many investigators, and the viability of mice which are homozygous null mutants for the gp130 receptor subunit common to all members of the LIF cytokine family, all strongly suggest that the regulation of pluripotent stem cells is complex and multifactorial. The social behaviour of candidate ES cells from humans and other species, including their requirement for feeder cell layers and their aversion to culture as single cells at low density, which usually leads to differentiation or death, likely reflects a complex array of intercellular interactions required for stem cell renewal.

It is known in the prior art that, in the peri-implantation embryo, the pluripotent cells of the inner cell mass will give rise to two closely apposed cell populations: the embryonic ectoderm which will form the tissues of the embryo proper, and the primitive endoderm which will become the yolk sac. It is now clear from experiments in the mouse that the latter plays an important role in regulating the growth and differentiation of the former.

A number of cultured cell lines have been derived from human testicular teratomas. Some of them reproduce the phenotype of embryonal carcinoma or yolk sac tumour seen in tumours in vivo, and a few show multipotentiality, the capacity for differentiation into range of somatic and extraembryonic cell types. However, the studies of such cell lines are in their infancy.

It would accordingly be a significant advance in the art if a factor or group of factors could be identified which could regulate the growth of pluripotential cells from humans and other species.

Accordingly it is an object of the present invention to overcome, or at least alleviate, one or more of the difficulties or deficiencies in relation to the prior art.

Accordingly, in a first aspect of the present invention there is provided a composition for growing pluripotential cells and/or for directing their differentiation, said composition including a factor or factors capable of inhibiting an activity of bone morphogenetic protein-2 (BMP-2).

Applicant has surprisingly found that BMP-2 induces differentiation of human pluripotent stem cells into a cell type with the properties of extraembryonic endoderm. Whilst applicant does not wish to be restricted by theory, it is thought that such differentiation may lead to extinction of stem cells in the culture, or limit stem cell differentiation into desired somatic cell lineages. Applicant has also surprisingly found that cultures of human pluripotent cells contain transcripts for BMP-2 receptors and for BMP-2 itself. Thus, it appears that an autocrine positive feedback loop exists in human pluripotent stem cell cultures which can limit stem cell renewal or inhibit differentiation into desired somatic cell lineages.

The composition may be of any suitable type. The composition may be a culture medium. The composition may include components such as a basal medium suitable for growth of ES or EG cells, foetal calf serum, nonessential amino acids, beta mercaptoethanol, and a mouse embryonic fibroblast feeder cell layer, the use of which is well known in the art. A person skilled in the art may readily determine the concentrations of such components in the composition. Temperature and other culture conditions may also be readily determined by a person skilled in the art. For example a temperature of approximately 37° C. and humidified atmosphere containing CO₂ at approximately 5-10% may be used.

The pluripotential cells may be of any suitable type, including ES cells and EG cells. The pluripotential cells may be reconstituted cells. The pluripotential cells may be from human or non-human mammals or other species. In certain aspects of the present invention the pluripotential cells may be from a mammal species other than mouse.

The factor or factors which inhibit the action of BMP-2 are herein referred to as BMP-2 antagonist(s). The BMP-2 antagonist(s) may be derived from primitive endoderm cell lines. Such cell lines may be, for example, derived from testicular teratocarcinomas, particularly human testicular teratomas. A particularly preferred cell line is primitive endoderm cell line GCT44, as hereinafter described. The BMP-2 antagonist(s) may be a novel factor(s). The BMP-2 antagonist(s) may be a soluble factor or a membrane bound factor shed into the medium by the primitive endoderm cells or an extracellular matrix component produced by the cells. The BMP-2 antagonist(s) may be a factor or combination of factors known from studies of animal embryology to inhibit the action of BMP-2, including but not limited to chordin, noggin, DAN and cerebrus. The BMP-2 antagonist may be a modified form of a BMP-2 receptor which is capable of binding BMP-2 but does not activate signal transduction pathways associated with BMP-2 induced differentiation. The BMP-2 antagonist may be a small molecule which interfers with the signal transduction pathway involved in BMP-2 induction of pluripotential cell differentiation.

The concentration of BMP-2 antagonist in the composition may be readily determined by a person skilled in the art depending on the BMP-2 antagonist to be used. In general terms, the BMP-2 antagonist is used at a dose determined to inhibit extraembryonic differentiation of EC or ES cells, preferably human EC or ES cells.

In a further aspect of the present invention, there is provided a method of regulating the growth and/or differentiation of pluripotential cells, said method including culturing said cells in the presence of a factor or factors capable of inhibiting an activity of BMP-2.

Preferably, the method includes

providing:
a pluripotential cell line, and
a culture medium including an effective amount of said factor or factors; and
culturing the cell line in the culture medium.

The culturing may be of any suitable type. Preferably the cells are cultured in a composition which includes components such as a basal medium suitable for growth of ES or EG cells, foetal calf serum, nonessential amino acids, beta mercaptoethanol, and a mouse embryonic fibroblast feeder cell layer, the use of which is well known in the art. A person skilled in the art may readily determine the concentrations of such components in the composition. Temperature and other culture conditions may also be readily determined by a person skilled in the art. For example a temperature of approximately 37° C. and humidified atmosphere containing CO₂ at approximately 5-10% may be used.

The pluripotential cells may be of any suitable type, including ES cells and EG cells. The pluripotential cells may be reconstituted cells. The pluripotential cells may be from human or non-human mammals or other species. In certain aspects of the present invention the pluripotential cells may be from a mammal species other than mouse.

The BMP-2 antagonist(s) may be derived from primitive endoderm cell lines. Such cell lines may be, for example, derived from testicular teratocarcinomas, particularly human testicular teratomas. A particularly preferred cell line is primitive endoderm cell line GCT44, as hereinafter described. The BMP-2 antagonist(s) may be a novel factor(s). The BMP-2 antagonist(s) may be a soluble factor or a membrane bound factor shed into the medium by the primitive endoderm cells or an extracellular matrix component produced by the cells. The BMP-2 antagonist(s) may be a factor or combination of factors known from studies of animal embryology to inhibit the action of BMP-2, including but not limited to chordin, noggin, DAN and cerebrus. The BMP-2 antagonist may be a modified form of a BMP-2 receptor which is capable of binding BMP-2 but does not activate signal transduction pathways associated with BMP-2 induced differentiation. The BMP-2 antagonist may be a small molecule which interfers with the signal transduction pathway involved in BMP-2 induction of pluripotential cell differentiation.

The concentration of BMP-2 antagonist in the composition may be readily determined by a person skilled in the art depending on the BMP-2 antagonist to be used. In general terms, the BMP-2 antagonist is used at a dose determined to inhibit extraembryonic differentiation of EC or ES cells, preferably human EC or ES cells.

It will be understood that the BMP-2 antagonist may be utilised either alone, or in combination with another factor(s). Accordingly, the composition and method of the present invention may further include a secondary factor.

The secondary factor may be of any suitable type. The secondary factor may be one or more of the polypeptides described above, or a biologically active agonist, antagonist, mutant, fragment or derivative thereof.

In a particular preferred aspect, the secondary factor may include a ligand or ligands. Ligands such as CD30 or the like, including functionally equivalent ligands, and/or ligands of the Notch family of receptors may be used.

By the term “functionally equivalent ligands” is meant ligands having one or more of the biological properties of CD30.

In a further aspect of the present invention there is provided a method for producing a factor or group of factors capable of antagonising an action of BMP-2 on pluripotential cells, said method including

providing
a cell line representative of primitive endoderm, and
a suitable culture medium; and
culturing the cell line in the culture medium for a period of time sufficient for said cell line to produce said factor, or group of factors.

By “a cell line representative of primitive endoderm” is meant an epithelial cell line which can give rise to cells showing expression of surface markers and genes consistent with that of parietal and visceral endoderm in the mouse embryo, such as the cell line GCT 44. Preferably the cell line representative of primitive endoderm is derived from testicular carcinomons, particularly human testicular teratomas. A particularly preferred cell line is the primitive endoderm cell line GCT 44, as hereinafter described, or a functionally similar cell line.

The factor or group of factors may be a soluble factor or a membrane bound factor shed into the medium by the cells or an extracellular matrix component produced by the cells.

The culture medium may be of any suitable type. The culture medium may be a serum free medium. A medium such as IMDM may be used. The medium may include antibiotics such as penicillin and streptomycin. The medium may also include albumin transferrin, insulin and/or glutamine or the like.

The culturing may continue over an extended period, e.g. approximately one to six weeks, more preferably approximately two to five weeks, most preferably approximately three to four weeks. The medium may be harvested periodically, for example, on a weekly basis.

In a preferred form of this aspect of the invention, the method may include the further step of subjecting the factor or group of factors so produced to a purification step.

The purification step may be of any suitable type. Purification techniques including tangential flow filtration, anionic exchange, cationic exchange and reverse phase chromatography or a combination thereof may be used. Such purification steps may be conducted sequentially or in any other order.

In a further preferred form of this aspect of the invention, the method may include the still further step of assaying for the factor or group of factors.

The assaying may be performed before, during or after purification, preferably during purification. The assaying may be performed utilising a bioassay, preferably a bioassay using pluripotential cells, e.g. human pluripotential cells. The bioassay may include utilising GCT type multipotent stem cells grown in the presence of BMP-2. BMP-2 antagonism may be detected by comparison of one or more of the following variables in control cells, cells grown in the presence of BMP-2, and cells grown in the presence of BMP-2 plus an antagonist: cell growth (for example tritiated thymidine incorporation), immunological assays for stem cell markers (for example, flow cytometry measurement of the proportion of stem cells in a culture using a monoclonal antibody e.g. monoclonal antibody GCTM-2); assays for stem cell specific gene expression (for example, determination of Oct-4 transcript levels e.g. using RT-PCR or Northern blot analysis); or assays for gene expression characterisitic of extraembryonic endoderm (for example, determination of HNF-3α transcript levels e.g. using RT-PCR or Northern blot analysis).

As will be understood from the above, the BMP-2 antagonist may represent a member of a novel family of BMP-2 antagonists.

In a still further aspect of the present invention there is provided a pluripotential cell or cell line or a differentiated cell or cell line produced using the composition or method of the present invention.

The pluripotential cells may be of any suitable type, including ES cells and EG cells. The pluripotential cells may be reconstituted cells. The pluripotential cells may be from human or non-human mammals or other species. In certain aspects of the present invention the pluripotential cells may be from a mammal species other than mouse. Preferably the pluripotential cells are from a species other than mouse.

The pluripotential cells of the present invention may be utilised in the production of embryos and animals, utilising known techniques, for example as described in PCT/US98/22882 “Nuclear Transfer for production of transgenic animal embryo”.

Accordingly in a further aspect of the present invention there is provided an embryo or animal, including transgenic embryos and animals, produced using the composition or the method of the present invention. The embryo or animal may be human or non-human.

The present invention will now be more fully described with reference to the accompanying examples. It should be understood, however, that the description following is illustrative only, and should not be taken in any way as a restriction on the generality of the invention described above.

In the figures:

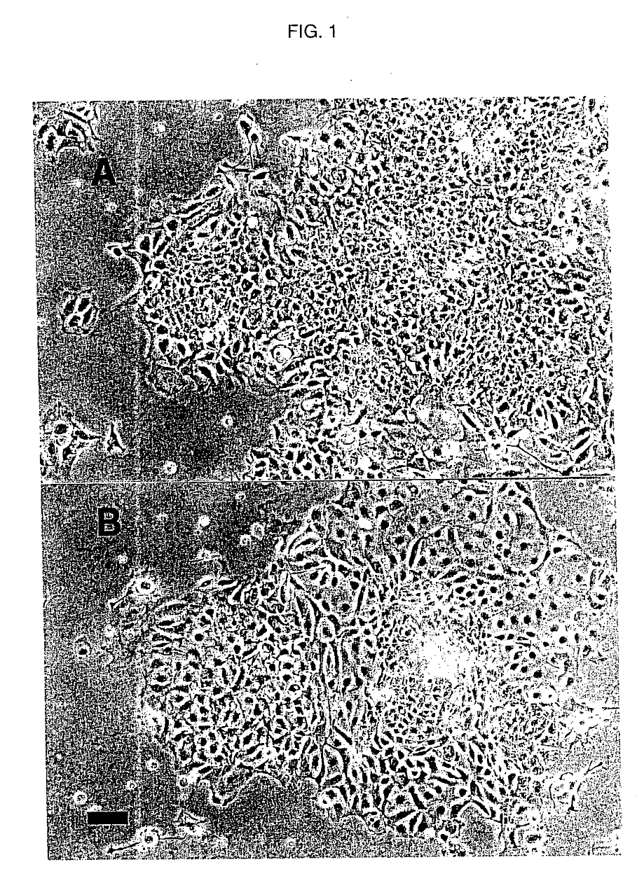

FIG. 1 shows morphology under phase contrast microscopy of control (A) and BMP-2 treated (B) GCT 27X-1 cells. Treatment was with 50 ng/ml BMP-2 and cells were photographed after three days of culture. Scale bar =10 microns.

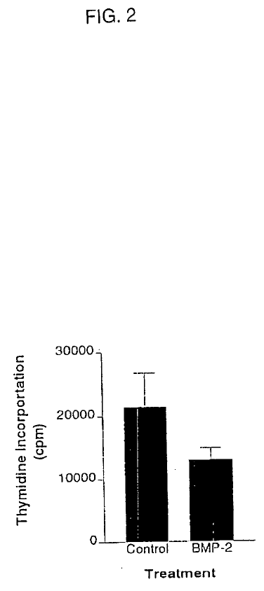

FIG. 2 shows the effect of BMP-2 treatment on 3H thymidine incorporation into GCT 27X-1 cells. Treatment was with 25 nm/ml BMP-2 for three days. Values are means +/− standard error of three wells.

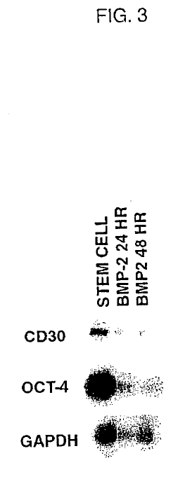

FIG. 3 shows the effect of BMP-2 treatment on levels of transcripts for CD30, Oct-4 and glyceraldehyde 3 phosphate dehydrogenase. Lanes show control cells and cells treated with 50 ng/ml BMP-2 for 24 or 48 hours respectively. Lanes contained approximately 2.5 ug of polyadenylated RNA.

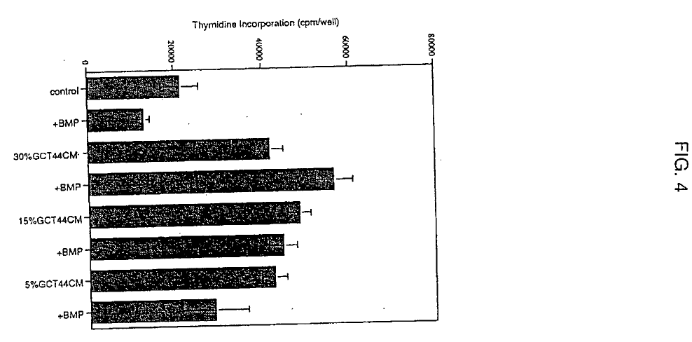

FIG. 4 shows the effect of BMP-2 treatment on 3H thymidine incorporation into GCT 27X-1 cells.

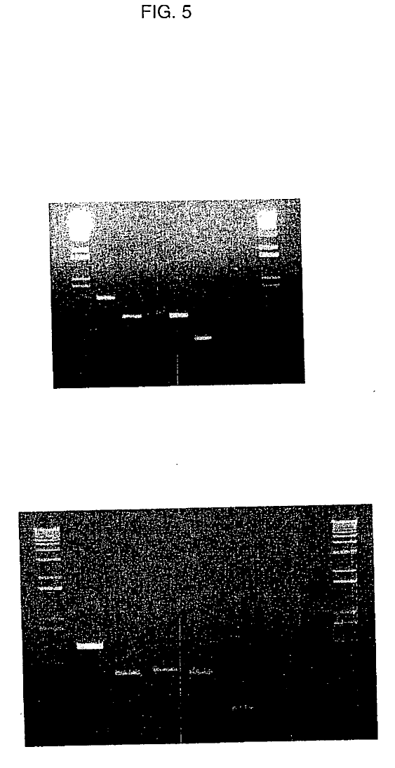

FIG. 5 shows the examination of BMP-2 and its receptors by RT-PCR. RT-PCR was used to determine the presence of BMP-2, BMP-2 Receptor Type IA (RIA), BMP-2 Receptor Type II (R II) and Activin Receptor Type IIB (Act R IIB) in the GCT 27X1 and GCT 44M1 cell lines.

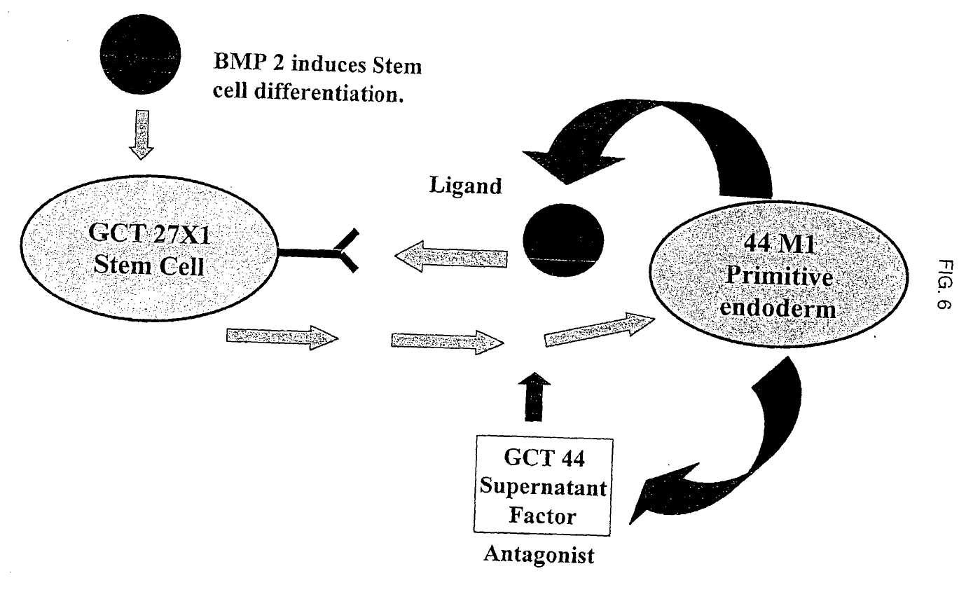

FIG. 6 shows the proposed positive feedback loop of BMP-2 induced stem cell differentiation.

### EXAMPLE 1

### Differentiation of Human Pluripotent Teratocarcinoma Stem Cells Induced by Bone Morphogenetic Protein-2

Introduction

During a screening of factors for the support of human EC cell growth (Roach et al., 1993), we noted that the TGF β superfamily member of BMP-2 had a strong effect on inducing differentiation. Here we describe this effect and report a preliminary characterisation of the differentiated cells.

Materials and Methods

Cell Culture and Induction of Differentiation:

Culture of EC cells was carried out as described (Pera et al., 1987, Pera et al., 1989). Treatment of GCT 27XI cells with test polypeptides was performed by continuous exposure of monolayer culture grown at high density from cell clumps harvested with dispase in the absence of a feeder cell layer (1:2-1:5 split), a procedure which suppresses spontaneous differentiation. Fresh peptides were added at 3-4 day intervals when the medium was changed. Cells were observed for a 14 day period on a daily basis.

Thymidine Incorporation:

The effect of BMP-2 on thymidine incorporation by GCT 27X-1 cells was studied by pulse labelling of cells grown in 6-well plates for three days in the presence or absence of the factor. Labelling with 0.20 Ci/ml 3H-thymidine (25-30 Ci./mmol) in standard growth medium was carried out for two hours, after which the cells were harvested and fixed as described (Heath, 1987) and the extracts counted in an aqueous scintillation fluid. Triplicate wells of control and treated cells were analysed. Results were expressed as cmp/well.

RNA Isolation and Analysis:

PolyA+ mRNA was isolated from cells (three 175 cm² flasks) prior to treatment and 24 and 48 hour post treatment with 50 ng/ml BMP-2 and subjected to Northern blot analysis as described (Roach et al., 1994). Probes included full length human CD30 cDNA (supplied by the Immunex Corporation), Oct-4 (mouse cDNA probe), and glyceraldehyde-3-phosphate dehydrogenase (Roach et al., 1994).

Immunofluorescence:

Indirect immunofluorescence analysis was carried out on control cells and cells treated with 50 ng/ml BMP-2 for 4 to 6 days as described elsewhere (Pera et al., 1989). Double labelling was performed to identify stem cells using the antibody GCTM-2 directed against the core protein of a pericellular matrix keratan sulphate/chondroitin sulphate proteoglycan. GCTM-2 was labelled with biotin and visualised with Texas Red Strepavidin, whilst the other antibodies were visualised with anti-mouse immunoglobulin conjugated to fluorescein.

Results

Screening of Polypeptides for Differentiation Activity:

GCT 27M cells were cultured at high density in the absence of a feeder cell layer, and the following proteins were added at concentrations between 1-200 ng/ml: BMP-2, BMP-4, BMP-5, BMP-6, BMP-7, BMP-9. No effects on cell growth or morphology were observed with any of the peptides tested apart from BMP-2. Treatment with this cytokine at doses between 20-200 ng/ml induce flattening and enlargement of the cell population (FIG. 1) and the effect was evidence after 48 hours of treatment. The enlarged cells shown in FIG. 1B no longer expressed the stem cell markers studied below. Although there were fewer cells in the treated cultures, the peptide did not appear to induce toxicity. The appearance of the treated cells was similar to that of GCT27X-1 cells treated with retinoic acid as described previously.

Effects of BMP-2 on Cell Growth:

For further studies of the action of BMP-2 on GCT27X-1 cells, we used a dose of 25 nm/ml since this was the lowest dose which consistently produced a uniform effect in cell differentiation. Following three days of growth, this does of BMP-2 reduced thymidine incorporation to a value about 60% that of the control cells (FIG. 2). However, inhibition of proliferation alone does not account for the action of BMP-2 on cell morphology or phenotype, since inhibition of proliferation by cultivation in reduced serum concentrations for example does not induce the changes seen with BMP-2.

Expression of Stem Cell Markers at RNA Level:

We studied expression of several markers of EC stem cells at the RNA level. In the mouse, the expression of the transcription factor Oct-4 is limited to pluripotent cells (review, Brehm et al., 1998). CD30 is a cell surface receptor of the tumor necrosis factor superfamily with a pattern of expression limited to EC stem cells and certain classes of lymphocytes in the T and B cell lineages (Latza, 1995; Pera et al., 1997). FIG. 3 shows a Northern blot of control cells and cells treated for 24 or 48 hours with BMP-2 at 50 μg/ml. Transcripts of the expected size for CD30 and Oct-4 were obtained from untreated human EC stem cells. Following treatment with BMP-2, CD30 RNA levels declined rapidly in treated GCT 27X-1 cells; levels of Oct-4 transcripts also declined. The decline in levels of CD30 transcript was generally more rapid than that of Oct-4 but levels of both transcripts were consistently reduced by 48 hours.

Effect of BMP-2 on Stem Cell and Differentiation Antigens:

Cells grown on glass multiwell slides were treated with BMP-2 at a dose of 25 ng/ml and their expression of a range of stem cell and differentiation markers was examined by indirect immunofluorescence (Table 1). Decreased expression of the following stem cell antigens was observed: GCTM-2, a stem cell pericellular matrix proteoglycan, CD30, a stem cell surface receptor, and GCTM-4, a stem cell marker confined to late endosomes/early endosomes. In agreement with the thymidine labelling data above, the proportion of cells staining with the proliferation marker Ki-67 was reduced though labelled cells were still seen. FIG. 4 shows a flow cytometry analysis of the proportion of cells stained with the stem cell marker GCTM-2 in control and BMP-2 treated cultures.

TABLE 1
Antigen expression in stem cells and BMP-2 treated cultures

Expression

| Antigen | Stem cell | BMP-2 |
|---|---|---|
| Stem cell specific markers | | |
| GCTM-2 | +++ | − |
| TRA1-60 | +++ | − |
| CD30 | +++ | − |
| GCTM-4 | +++ | − |
| Other markers | | |
| Cytokeratin 8, 18 | +++ | +++ |
| Desmoplakin | ++ | +++ |
| Cytokeratin 19 | + | +++ |
| GCTM-3 | + | +++ |
| Vimentin | + | +++ |
| KI-67 | +++ | ++ |
| Fibronectin | + | +++ |
| Type IV collagen | + | +++ |
| HNF3-α | − | +++ |
| Placental Alkaline Phosphatase | + | − |
| W6/32 | − | − |

− no expression
+ expression in a minority of cells (<10%)
++ expression in many cells (10-80%)
+++ expression in most cells (>80%)

Several markers characteristic of epithelial cells were strongly expressed by both treated and control cells, including cytokeratins 8 and 18 and desmoplakins 1 and 2.

The treated cells also strongly expressed cytokeratin 19, though controls did not. Treated cells also showed much stronger expression of vimentin intermediate filaments and the cytoskeletal antigen recognised by monoclonal antibody GCTM-3.

The nuclei of the differentiated cells were stained with antisera directed against hepatocytic nuclear factor 3-α. In the treated cultures, we observed extensive deposition of extracellular fibrous material stained with antibodies to tpe IV collagen or fibronectin.

The treated cells were not stained with antibodies to placental alkaline phosphatase, nor with antibodies to class I MHC antigen.

Discussion

BMP-2 treatment slowed the growth of human EC cells and decreased their expression of transcripts for the stem cell markers CD30 and OCT-4. Immunofluorescence labelling indicated that the treated cells expressed many markers in common with yolk sac carcinoma cells representing an early stage of endoderm differentiation including cytoskeletal antigens, extracellular matrix proteins and HNF-α. This pattern of marker expression induced by BMP-2 treatment is very similar to that previous observed for retinoic acid. The pattern is consistent with differentiation into extraembryonic endoderm, though the paucity of markers for early stages of differentiation in this cell lineage makes it difficult to identify the cell unequivocally. Clearly the differentiated cells are epithelial in nature and express some markers characteristic of endoderm. The fact that they do not express MHC Class. I antigens on their surface argues against their identification as somatic endodermal cells.

### EXAMPLE 3

The graphs in FIG. 4 show the inhibition effects of BMP-2 by conditioned medium from a yolk sac carcinoma cell line.

### EXAMPLE 4

### Regulation of Human Pluripotent Stem Cell Differentiation by Bone Morphogenetic Protein-2

The previous examples demonstrate that exogenous BMP-2 can induce human pluripotent stem cells to differentiate into cells with the property of extraembryonic endoderm. BMP-2 treatment of pluripotent human embryonal carcinoma cell line GCT 27M results in decreased expression of the stem cell markers CD30, GCTM-2 cell surface proteoglycan, and Oct-4, and an enhanced expression of markers characteristic of early stages of extraembryonic endoderm differentiation. The treatment converts the entire culture to a cell population with these properties. Since this type of differentiation is also seen spontaneously, we investigated whether an autocrine positive feedback loop might be operating to drive stem cell differentiation into the extraembryonic lineage.

RT-PCR analysis showed that GCT-27X-1 stem cells express transcripts for BMP Receptor IA and III, Activin receptor IIB, and BMP-2 itself (FIG. 5 ). Using degenerate primers designed to amplify coding regions conserved across members of TGF-beta superfamily, we found that human yolk sac carcinoma cell line GCT 44, which resembles primitive endoderm, expresses transcripts for BMP-2 and BMP-4. However, conditioned medium from this cell line can also antagonise the growth inhibitory effects of exogenous BMP-2 on human embryonal carcinoma stem cells. Our results are consistent with a model in which BMP-2 drives spontaneous differentiation of human pluripotent stem cells towards the extraembryonic endoderm lineage in a positive feedback loop. Endoderm production of factors which counteract this effect may be necessary in the embryo to prevent premature extinction of stem cells or to enable spatial patterning of other differentiation lineages under the direction of endoderm. In practical terms, blockade of this endogenous differentiation pathway may enhance the growth of human pluripotent stem cells in vitro, or facilitate directed differentiation of the cells into desired somatic lineages.

The above findings show that human EC cells themselves express human BMP-2 as well as receptors for this protein. This latter finding suggests the possibility that BMP-2 may be an endogenous inducer of human pluripotent stem cell differentiation and leads us to conclude that antagonism of this pathway may be beneficial in culture of human pluripotent stem cells (FIG. 6).

### EXAMPLE 5

### GCT 44 Cell Line

GCT 44 was derived from a human testicular teratoma using methods described in Pera, 1987, the entire disclosure of which. is incorporated herein by reference. The methods may be summarised as follows.

A fresh biopsy specimen of a germ cell tumour containing yolk sac elements was dissected, then subjected to disaggregation in a solution containing coliagenase plus culture medium supplemented with foetal calf serum. The resulting digest was plated out on to a mouse embryonic fibroblast feeder cell layer in Dulbeccois Modified Eagles Medium supplemented with 10% foetal calf serum, glutamine, and antibiotics. When the clumps of tumour cells attached to the monolayer had begun to near confluence, the monolayer was treated briefly with a solution of trypsin and ethylenediamminetetraacetic acid to release clumps which were replated at a 1:3 split ratio onto a new feeder cell layer. The cell line was expanded and maintained in this fashion and cryopreserved.

Characterisation of the cell line was carried out by examining expression of surface and cytostructural antigens, assay for secreted products, assay for gene expression using Northern blot analysis or RT-PCR, and formation of tumours in immunosuppressed mice, and by assessment of the ability of supernatants from the cell line to support growth of pluripotent human embyonal carcinoma cells as described in Pera, 1989.

A primitive endoderm cell line is distinguished from other types of cells derived from human germ cell tumours by its proflie of antigen and gene expression, and by the histology of tumours formed in immunodeprived mice, as described in Pera, 1987, and Roach, 1994.

### Bibliography

Brehm, A., Ovitt, C. E., and Scholer, II. R. (1998). Oct-4: more than just a POUerful marker of the mammalian germine? APMIS 106, 114-126.
Heath, J. K. (1987). Experimental analysis of teratocarcinoma cell multiplication and purification of embryonal carcinoma derived growth factor. In Teratocarcinomas and embryonic stem cells: a practical approach. E. J. Robertson, ed. (Oxford: IRL Press), pp.183-206.
Latza, U., Foss, H. D., Durkop, H., Eitelbach, F., Dieckmann, K. P., Loy, V., Unger, M., Pizzolo, G., and Stein, H. (1995). CD30 antigen in embryonal carcinoma and embryogenesis and release of the soluble molecule. Am. J. Pathol. 146, 463-471.
Pera, M. F., Bennett., W., and Cerretti, D. P. (1997). Expression of CD30 and CD30 ligand in cultured cell lines from germ cell tumours. Laboratory Investigation 76, 497-504.
Pera, M. F., Cooper, S., Mills, J., and Parrington, J. M. (1989), Isolation and characterization of a multipotent clone of human embryonal carcinoma-cells. Differentiation 42, 10-23.
Pera et al, International Journal of Cancer, 40, 334-343 (1987), Cultured Stem-Cells from Human Testicular Teratomas: The Nature of Human Embryonal Carcinoma, and its Comparison with Two Types of Yolk-Sac Carcinoma.
Roach, S., Cooper, S., Bennett, W., and Pera, M. F. (1993). Cultured cell lines from human germ cell tumours: windows into tumour growth and differentiation and early human development. European Urology 23, 82-88.
Roach, S., Schmid, W., and Pera, M. F. (1994). Hepatocytic transcription factor expression in human embryonal carcinoma and yolk-sac carcinoma cell-lines—expression of hnf-3-alpha in models of early endodermal cell-differentiation. Exp. Cell Res. 215,189-198.

Finally it is to be understood that various alterations, modifications and/or additions may be made without departing from the spirit of the present invention as outlined herein.

1. A composition for growing pluripotential cells and/or for directing their differentiation, said composition including a factor or factors capable of inhibiting an activity of bone morphogenetic protein-2 (BMP-2).

2. A composition according to claim 1 wherein said pluripotential cells are embryonic stem (ES) or embryonic germ (EG) cells.

3. A composition according to claim 2 wherein said ES or EG cells are human ES or EG cells.

4. A composition according to claim 1 wherein said factor is derived from a primitive endoderm cell line.

5. A composition according to claim 4 wherein said factor is selected from the group consisting of a soluble factor or membrane bound factor shed by said cell line or an extra-cellular matrix component produced by said cell line.

6. A composition according to claim 5 wherein said primitive endoderm cell line is derived from a testicular teratocarcinoma.

7. A composition according to claim 6 wherein said cell line is primitive endoderm cell line GCT 44, as hereinbefore described.

8. A composition according to claim 1 wherein said factor is selected from the group consisting of chordin, noggin, DAN, cerebrus, a modified BMP-2 receptor which is capable of binding BMP-2 but does not activate signal transduction pathways associated with BMP-2 induced differentiation, and small molecules which interfere with signal transduction pathways involved in BMP-2 induction of pluripotential cell differentiation.

9. A composition according to claim 1 wherein said factor is present in said composition in a concentration capable of inhibiting extraembryonic differentiation of EC or ES cells.

10. A composition according to claim 1 further including a secondary factor, said secondary factor including a ligand or ligands.

11. A composition according to claim 10 wherein said ligand is selected from the group consisting of CD30 and functionally equivalent molecules and ligands of the Notch family of receptors.

12. A method of regulating growth and/or differentiation of pluripotential cells, said method including culturing said cells in the presence of a factor or factors capable of inhibiting an activity of BMP-2.

13. A method according to claim 12 including
providing
a pluripotential cell line, and
an effective amount of said factor; and
culturing the cell line in the culture medium.

14. A method according to claim 13 wherein said pluripotential cells are embryonic stem (ES) or embryonic germ (EG) cells.

15. A method according to claim 14 wherein said ES or EG cells are human ES or EG cells.

16. A method according to claim 13 wherein said factor is derived from a primitive endoderm cell line.

17. A method according to claim 16 wherein said factor is selected from the group consisting of a soluble factor or membrane bound factor shed by said cell line or an extra-cellular matrix component produced by said cell line.

18. A method according to claim 17 wherein said primitive endoderm cell line is derived from a testicular teratocarcinoma.

19. A method according to claim 18 wherein said cell line is primitive endoderm cell line GCT 44, as hereinbefore described.

20. A method according to claim 13 wherein said factor is selected from the group consisting of chordin, noggin, DAN, cerebrus, a modified BMP-2 receptor which is capable of binding BMP-2 but does not activate signal transduction pathways associated with BMP-2 induced differentiation, and small molecules which interfere with signal transduction pathways involved in BMP-2 induction of pluripotential cell differentiation.

21. A method according to claim 13 wherein said factor is present in the culture medium in a concentration capable of inhibiting extraembryonic differentiation of EC or ES cells.

22. A method according to claim 13 further including a secondary factor, said secondary factor including a ligand or ligands.

23. A method according to claim 22 wherein said ligand is selected from the group consisting of CD30 and functionally equivalent molecules and ligands of the Notch family of receptors.

24. A method for producing a factor or group of factors capable of antagonising an action of BMP-2 on pluripotential cells, said method including
providing
a primitive endoderm cell line, and
a suitable culture medium; and
culturing the cell line in the culture medium for a period of time sufficient to produce said factor.

25. A method according to claim 24 wherein said factor is selected from the group consisting of a soluble factor or membrane bound factor shed by said cell line or an extra-cellular matrix component produced by said cell line.

26. A method according to claim 25 wherein said primitive endoderm cell line is derived from a testicular teratocarcinoma.

27. A method according to claim 26 wherein said cell line is primitive endoderm cell line GCT 44, as hereinbefore described.

28. A method according to claim 24 wherein said suitable culture medium is a serum-free medium.

29. A method according to claim 28 wherein said medium is IMDM.

30. A method according to claim 24 wherein said period of time is approximately 3 to 4 weeks.

31. A method according to claim 24 further including subjecting the factor so produced to a purification step.

32. A method according to claim 31 wherein said purification step is selected from the group consisting of tangential flow filtration, anionic exchange, cationic exchange, reverse phase chromatography and combinations thereof.

33. A method according to claim 31 further including assaying for the factor so produced.

34. A method according to claim 33 wherein said assaying is performed during said purification step.

35. A method according to claim 33 wherein said assaying is performed using a bioassay using GCT type multipotent cells grown in the presence of BMP-2.

36. A pluripotential cell or cell line or a differentiated cell or cell line produced using the composition of claim 1.

37. A pluripotential cell or cell line or a differentiated cell or cell line produced by the method of claim 12.

38. An embryo or transgenic embryo derived from the cells of claim 36 or 37.

39. An animal or transgenic animal produced from the embryo or transgenic embryo of claim 38.
